(19) United States
(12) Patent Application Publication
(10) Pub. No.: US 2025/0381079 A1
(43) Pub. Date: Dec. 18, 2025

(54) FEMININE HYGIENE PAD

(71) Applicants: Linda Papadatis, Astoria, NY (US); Katerina Papadatis, Astoria, NY (US); Eleni Tropaitis, Astoria, NY (US)

(72) Inventors: Linda Papadatis, Astoria, NY (US); Katerina Papadatis, Astoria, NY (US); Eleni Tropaitis, Astoria, NY (US)

(21) Appl. No.: 18/740,941

(22) Filed: Jun. 12, 2024

(51) Int. Cl. A61F 13/475; A61F 13/56

(52) U.S. Cl. CPC A61F 13/4753; A61F 13/5616

(57) ABSTRACT

A feminine hygiene pad defined by a unitary, substantially planar body member and a pair of attachment flaps. The body member includes an elongated main section and a widened end section, a pair of edge ridges, and has a bottom liner suitable to be placed against and secured to a woman's undergarment and a pad portion disposed on top of the bottom surface that is suitable to absorb fluids which come into contact with it. The pair of attachment flaps extend outwardly from opposing edges of the body member and include both absorptive material and an attachment surface which enables them to removably attach to a woman's undergarment when the body is placed thereon. Through this design, the feminine hygiene pad offers an increased absorptive capability which provides enhanced protection for a seated or lying user while remaining invisible or minimally visible through the user's clothing.

### BACKGROUND OF THE INVENTION

### Field of the Invention

This invention relates generally to hygiene accessories and, more particularly, to a removably attachable hygiene pad for women having an increased protection capacity.

### Description of the Prior Art

The use and design of a conventional menstrual pad (or “pad,” “sanitary napkin,” or “feminine pad”) as a feminine hygiene product for use by a woman during menstruation, vaginal discharge, and other bodily functions related to the vagina is well established. Typically, pads are designed to be positioned between a woman's vulva and a woman's undergarments. Because this requires it to be positioned in an area between the woman's legs, such a typical pad will have a generally elongated shape which will not affect the ability of a woman to walk when in place. In addition, a typical pad will include absorbent material to absorb a flow of blood from a woman's vagina and prevent leaking, as well as some type of attachment material which allows it to be removably secured in place on the woman's undergarments.

A problem which still exists with typical pad designs is that while their generally elongated shape is needed for it to be positioned in an area flanked by the wearer's legs, it places inherent limitations on the absorbent capabilities of a given pad. This problem may be particularly acute when a woman is seated or lying down, as in such circumstances, the fluid needing to be absorbed is typically not going to be exposed to the surface of the pad evenly but rather primarily being directed towards the surface at one end of the pad. Thus, there remains a need for a pad for improving feminine hygiene outcomes that includes at one end a section that widens so as to increase absorbent capabilities at that end. It would additionally be desirable for such a feminine hygiene pad to include a pair of longitudinal edge ridges which also include absorbent material integrated therein. It would further be desirable for the wider end section of such a feminine hygiene pad to include a tapering section where it gradually becomes thinner from a depth perspective towards the edge of the pad to reduce visibility of the pad.

### SUMMARY OF THE INVENTION

The present disclosure provides for a feminine hygiene pad, comprising: a body member having a front end, rear end, and two longitudinal sides, wherein the body member includes an elongated main section and a widened end section, with the main section extending from the front end towards the rear end up to the end section and the end section extending from the rear end towards the front end up to the main section; a pair of edge ridges integral with the body member; a pair of attachment flaps integral with the body member, wherein each of the attachment flaps extends from one of the longitudinal sides of the body member; wherein the width of the body member from one longitudinal side to the other longitudinal side is greater in the end section than in the main section; wherein the body member includes a bottom liner forming the entire bottom surface of the body member and the attachment flaps each include a bottom flap liner forming the entire bottom surface of each of the attachment flaps; and wherein bottom liner includes attachment surfacing that is configured to releasably attach to fabric and the bottom flap liners each include flap attachment surfacing that is configured to releasably attach to fabric.

It is an object of the present disclosure to provide a feminine hygiene pad which includes at one end an end section which widens so as to increase absorbent capabilities at that end.

It is another object of the present disclosure to provide a feminine hygiene pad that includes a pair of edge ridges which also include absorbent material integrated therein.

It is an additional object of the present disclosure to provide a feminine hygiene pad which includes a wider end section which gradually becomes thinner from a depth perspective towards the edges of the pad to reduce visibility of the pad.

### BRIEF DESCRIPTION OF THE DRAWINGS

FIG. 1 is a top rear perspective view of a feminine hygiene pad built in accordance with the present invention.

FIG. 2 is a bottom rear perspective view of a feminine hygiene pad built in accordance with the present invention.

FIG. 3 is a side elevational view of a longitudinal cross section of the body member of a feminine hygiene pad built in accordance with the present invention.

FIG. 4 is a front elevational view of a transverse cross section of the body member of a feminine hygiene pad built in accordance with the present invention.

FIG. 5 is a front perspective view of a feminine hygiene pad built in accordance with the present invention shown in conjunction with a woman's undergarment.

FIG. 6 is an exposed bottom rear view of a feminine hygiene pad built in accordance with the present invention shown attached to a woman's undergarment.

### DETAILED DESCRIPTION OF THE INVENTION

Described herein is a feminine hygiene pad defined by a unitary, substantially planar body member and a pair of attachment flaps. The body member includes an elongated main section and a widened end section, and has a bottom liner suitable to be placed against and secured to a woman's undergarment, an absorbent layer disposed on top of the bottom liner that is suitable to absorb fluids which come into contact with it, and a porous top liner operative to secure the absorbent layer in place on the bottom liner. The absorbent layer and the top liner together form a pad portion. The pair of foldable attachment flaps extend outwardly from opposing edges of the body member and are formed from both top liner material and bottom liner material. Through this design, the feminine hygiene pad offers an increased absorptive capability which provides enhanced protection for a seated or lying user while remaining invisible or minimally visible through the user's clothing.

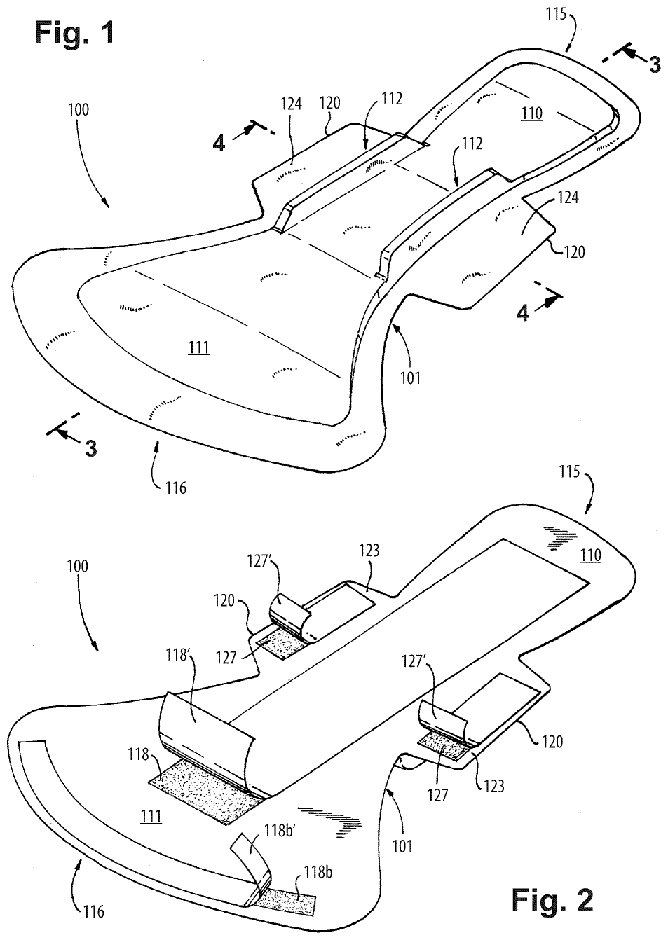

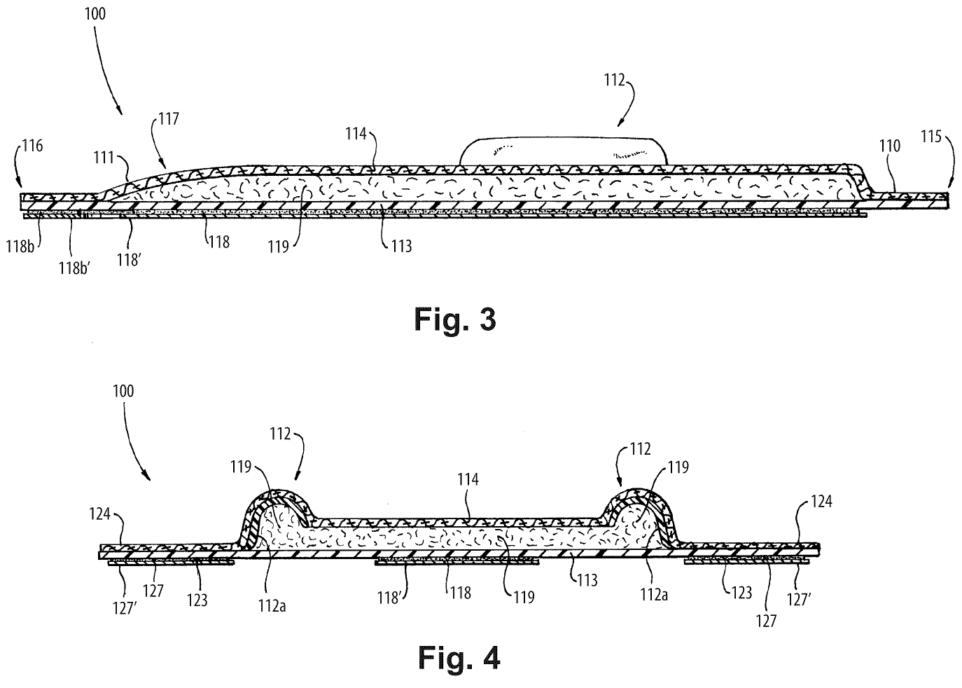

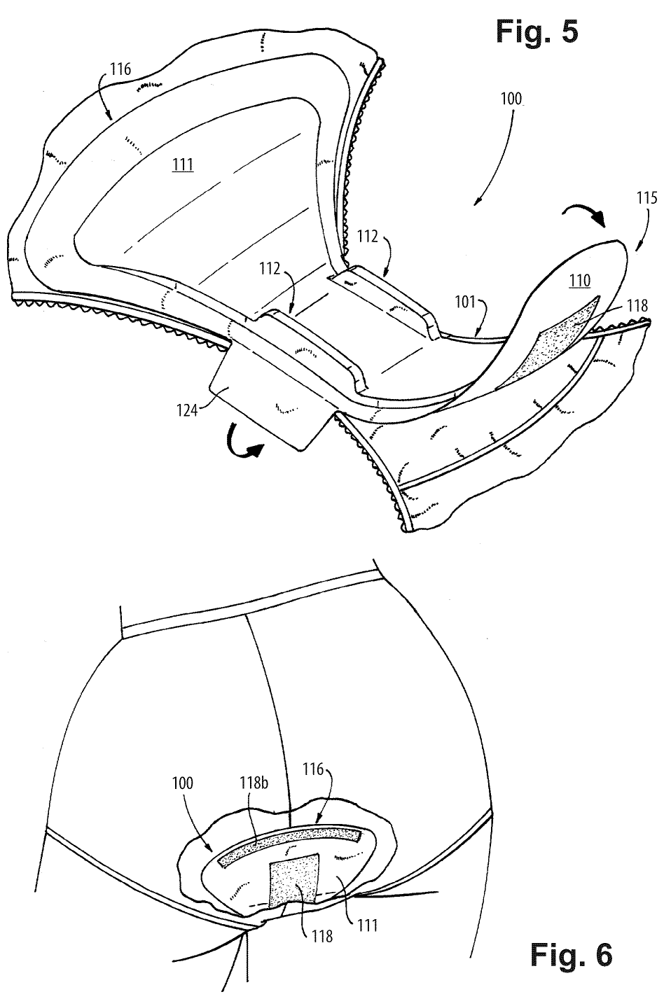

Referring now to the drawings and, in particular, FIGS. 1, 2, 3, 4, 5, and 6, a feminine hygiene pad 100 may be formed from a unitary body member 101 that is substantially planar and has an elongated main section 110 and a widened end section 111, as well as a pair of attachment flaps 120 which extend outwardly from the main section 110 of the body member 101. The body member 101 includes a smooth bottom liner 113 that has a porous top liner 114 disposed on top of it. The body member 101 additionally includes an absorbent layer 119 in between the bottom liner 113 and top liner 114. In the preferred embodiment, the bottom liner 113 and top liner 114 extend beyond the perimeter of the absorbent layer 119 where they are attached to each other, thus fully encapsulating the absorbent layer 119. In one embodiment, the portions of the bottom liner 113 and the top liner 114 that extend past the outer edges of the absorbent layer 119 are of substantially uniform width around the entire body member 101, not including the attachment flaps 120.

The bottom liner 113, top liner 114, and absorbent layer 119 may be constructed of conventional materials for menstrual pads. In this regard, the bottom liner 113 may be made of a single continuous sheet of thin and flexible plastic that is fluid impervious, or other flexible and fluid impervious material and the top liner 114 and the absorbent layer 119 may be constructed of a porous material such as bleached rayon, cotton, wood pulp, hemp, bamboo, and/or plastics. The absorbent layer 119 may additionally include powders or other granules which turn into a gel when absorbing fluids. It is additionally contemplated that the top liner 114 and/or absorbent layer 119 may include fragrance materials and/or antibacterial agents integral therewith. Any element of the feminine hygiene pad 100 may be constructed of natural materials, synthetic materials, or a combination of natural and synthetic materials.

The main section 110 may be formed as a narrow strip which extends from a front end 115 of the body member 101 to the end section 111. The end section 111 is defined as the portion of the body member 101 beginning where the main section 110 ends and the body member 101 begins to gradually widen and extending until a rear end 116 of the body member 101 that is opposite the front end. It is contemplated that the end section 111 may curve as it gradually widens so as to allow it to more easily contour with the buttocks of a wearer, as appreciated in FIG. 6. It is additionally contemplated that the entire main section 110 may be of the same width or may contour inwards and the widest part of the end section 111 may be at least twice as wide as the main section 110.

The main section 110 additionally includes a pair of edge ridges 112 which extend longitudinally along, and contour to the shape of, opposing longitudinally side edges of the main section 110. The edge ridges 112 have a similar layered construction as the rest of the main section 110, sharing the same absorbent layer 119 between the porous top liner 114 and fluid impervious bottom liner 113, but its much thicker absorbent layer 119 causes it to rise higher above all other parts of the top surface of the main section 110. The edge ridges 112 do, however, include a fluid impervious boundary portion 112a which may be constructed out of the same material as the bottom liner 113 and which operates to block any liquid from spilling sideways out from between the two edge ridges 112. As shown in FIG. 4, each boundary portion 112a preferably covers the entirety of the edge ridge 112, beginning at the bottom of the outer side edge of the main section 110 adjacent the bottom liner 113, and extending all the way around the edge ridge 112 to the portion of the top surface of the absorbent layer 119 not having the edge ridges 112. In an alternate embodiment, each boundary portion 112a begins at the bottom of the outer side edge of the main section 110 adjacent the bottom liner 113, and extends over the peak of the edge ridge 112 but ends at a location halfway between the highest point of the edge ridge 112 and the portion of the top surface of the absorbent layer 119 not having the edge ridges 112. In yet another embodiment, each boundary portion 112a begins at the bottom of the outer side edge of the main section 110 adjacent the bottom liner 113, and extends over the peak of the edge ridge 112 and past the location halfway between the highest point of the edge ridge 112 and the portion of the top surface of the absorbent layer 119 not having the edge ridges 112, but ends before reaching the portion of the top surface of the absorbent layer 119 not having the edge ridges 112. Since the top liner 114 may not be needed on top of portions of the absorbent layer 119 that are covered by the boundary portion 112a, the top liner 114 may not extend outward past such portions. Thus, in some embodiments, the top liner 114 does not cover the entire absorbent layer 119.

Other than the edge ridges 112, the body member 101 may otherwise be of uniform thickness throughout except in a tapering section 117 that is adjacent to the rear end 116, where it may taper and get progressively thinner as it gets closer to the rear end 116. The tapering section 117 allows the end section 111 to be less likely to create a bulge that shows through a wearer's clothing despite its increased width.

The bottom liner 113 may include attachment surfacing 118 and 118b on its bottom surface, running along the body member 101. As depicted in FIG. 2, the preferred embodiment includes attachment surfacing 118 running longitudinally down the center of the bottom liner 113. Moreover, the bottom liner 113 preferably includes attachment surfacing 118b on its bottom surface underneath the portion of the end section 111 that is adjacent to the rear end 116. Such attachment surfacing 118b may partially or fully cover the tapering section 117. The attachment surfacing 118 and 118b is configured to releasably attach to fabric, and may be defined by an adhesive substance in place on the bottom liner 113. It is appreciated that this attachment surfacing 118 and 118b may work along with the structure of the attachment flaps 120 when folded down to provide a more distributed attachment force that is able to hold the feminine hygiene pad 100 in place even when faced with shear and other forces from a seated or lying wearer which could cause the feminine hygiene pad 100 to reposition itself if it were only secured by the attachment flaps 120. In particular, the attachment surfacing 118b adjacent to the rear end 116 is especially helpful in preventing the feminine hygiene pad 100 from wrinkling or bunching up during use. The attachment surfacing 118 and 118b may be covered with a removable protective covering 118′ and 118b′, respectively, that may be peeled away before attaching the attachment surfacing 118 and 118b to an undergarment.

Each of the attachment flaps 120 extend outwardly from the edge of one of the longitudinal sides of the body member 101, particularly the main section 110 of the body member 101. The attachment flaps 120 may be constructed in substantially the same manner as the body member 101, and may include both a bottom flap liner 123 having the same structural properties as the bottom liner 113 and a top flap liner 124 having the same structural properties as the top liner 114. The bottom flap liner 123 may include flap attachment surfacing 127 on its bottom surface. The flap attachment surfacing 127 is configured to releasably attach to fabric, and may be defined by an adhesive substance in place on the bottom flap liner 123. The flap attachment surfacing 127 may be covered with a removable flap protective covering 127′ that may be peeled away before attaching the flap attachment surfacing 127 to an undergarment.

The top flap liner 124 preferably provides the attachment flaps 120 with fluid absorptive material which can catch any fluid that make leak over the edge of the feminine hygiene pad 100 or a women's undergarment to which it has been coupled.

In use, the feminine hygiene pad 100 is to be positioned in the crotch area of a target women's undergarment and then secured in place. The body member 101 may be secured in place first by placing the feminine hygiene pad 100 in its desired location and pressing the part of the body member 101 down into the interior of the target undergarment. It is appreciated that this action will cause the attachment surfacing 118 and 118b on the bottom liner 113 to removably attach to the target undergarment. The attachment flaps 120 can then be folded down until their flap attachment surfacing 127 on their bottom flap liners 123 contacts the bottom exterior surface of the target undergarment. It is appreciated that this action will cause the flap attachment surfacing 127 of each bottom flap liner 123 to removably attach to the target undergarment.

It is appreciated that the feminine hygiene pad 100 can be detached from the target undergarment by simply reversing this action, namely folding up the attachment flaps 120 until the flap attachment surfacing 127 detaches and lifting the body member 101 from the interior of the target undergarment until the attachment surfacing 118 and 118b detaches from the target undergarment.

Embodiments of the feminine hygiene pad may be constructed with attachment flaps 120 that each include a hinging section defined by a portion of bottom flap liner 123 in between the top flap liner 124 and the body member 101. In such an embodiment, the attachment flaps 120 may be structured such that the top flap liner 124 does not extend over the hinging section. In this regard, the hinging section may make it easier to swing the attachment flaps 120 into place when it is desired to attach the feminine hygiene pad to a women's undergarment.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A feminine hygiene pad, comprising:
a body member having a front end, rear end, and two longitudinal sides, wherein the body member includes an elongated main section and a widened end section, with the main section extending from the front end towards the rear end up to the end section and the end section extending from the rear end towards the front end up to the main section;
a pair of edge ridges integral with the body member, wherein each of the edge ridges is defined by a raised surface running alongside one of the longitudinal sides of the body member;
a pair of attachment flaps integral with the body member, wherein each of the attachment flaps extends from one of the longitudinal sides of the body member;
wherein the width of the body member from one longitudinal side to the other longitudinal side is greater in the end section than in the main section;
wherein the body member includes a bottom liner forming the entire bottom surface of the body member and the attachment flaps each include a bottom flap liner forming the entire bottom surface of each of the attachment flaps; and
wherein the bottom liner includes attachment surfacing that is configured to releasably attach to fabric and the bottom flap liners each include flap attachment surfacing that is configured to releasably attach to fabric.

2. The feminine hygiene pad of claim 1, wherein the body member includes a pad portion positioned above the bottom liner and the attachment flaps include a top flap liner positioned above the bottom flap liner.

3. The feminine hygiene pad of claim 2, wherein:
the pad portion is defined by a top liner and an absorbent layer; and
the body member includes a tapering section adjacent to the rear end.

4. The feminine hygiene pad of claim 1, wherein the end section includes a portion of the body member where the width of the body member incrementally increases.

5. The feminine hygiene pad of claim 1, wherein in the end section, the longitudinal sides of the body member are curved.

6. The feminine hygiene pad of claim 1, wherein in the main section, the longitudinal sides of the body member are curved.

7. The feminine hygiene pad of claim 1, wherein the attachment surfacing on the bottom liner extends longitudinally along the bottom liner.

8. The feminine hygiene pad of claim 1, wherein the width of the body member from one longitudinal side to the other longitudinal side is greater at all parts of the end section.

9. The feminine hygiene pad of claim 1, wherein the attachment flaps are mirror images of one another.

10. The feminine hygiene pad of claim 1, wherein the bottom liner and the bottom flap liners are together formed from a single, continuous sheet of thin and flexible material.

11. A feminine hygiene pad, comprising:
a body member having a front end, rear end, and two longitudinal sides, wherein the body member includes an elongated main section and a widened end section, with the main section extending from the front end towards the rear end up to the end section and the end section extending from the rear end towards the front end up to the main section;
a pair of edge ridges integral with the body member, wherein each of the edge ridges is defined by a raised surface running alongside one of the longitudinal sides of the body member;
a pair of attachment flaps integral with the body member, wherein each of the attachment flaps extends from one of the longitudinal sides of the body member;
wherein the width of the body member from one longitudinal side to the other longitudinal side is greater in the end section than in the main section and the end section includes a portion of the body member where the width of the body member incrementally increases;
wherein the body member includes a bottom liner forming the entire bottom surface of the body member and the attachment flaps each include a bottom flap liner forming the entire bottom surface of each of the attachment flaps;
wherein the bottom liner includes attachment surfacing that is configured to releasably attach to fabric and the bottom flap liners each include flap attachment surfacing that is configured to releasably attach to fabric; and
wherein the body member includes a pad portion positioned above the bottom liner and the attachment flaps include a top flap liner positioned above the bottom flap liner.

12. The feminine hygiene pad of claim 11, wherein:
the pad portion is defined by a top liner and an absorbent layer; and
the body member includes a tapering section adjacent to the rear end.

13. The feminine hygiene pad of claim 11, wherein in the end section, the longitudinal sides of the body member are curved.

14. The feminine hygiene pad of claim 11, wherein in the main section, the longitudinal sides of the body member are curved.

15. The feminine hygiene pad of claim 11, wherein the attachment surfacing on the bottom liner extends longitudinally along the bottom liner.

16. The feminine hygiene pad of claim 11, wherein the attachment surfacing on the bottom liner extends along a section adjacent to the rear end.

17. The feminine hygiene pad of claim 11, wherein the width of the body member from one longitudinal side to the other longitudinal side is greater at all parts of in the end section.

18. The feminine hygiene pad of claim 11, wherein the attachment flaps are mirror images of one another.

19. The feminine hygiene pad of claim 11, wherein the bottom liner and the bottom flap liners are together formed from a single, continuous sheet of thin and flexible material.

20. A feminine hygiene pad, comprising:
a body member having a front end, rear end, and two longitudinal sides, wherein the body member includes an elongated main section and a widened end section, with the main section extending from the front end towards the rear end up to the end section and the end section extending from the rear end towards the front end up to the main section;
a pair of edge ridges integral with the body member, wherein each of the edge ridges is defined by a raised surface running alongside one of the longitudinal sides of the body member;
a pair of attachment flaps integral with the body member, wherein each of the attachment flaps extends from one of the longitudinal sides of the body member;
wherein the width of the body member from one longitudinal side to the other longitudinal side is greater in the end section than in the main section and the end section includes a portion of the body member where the width of the body member incrementally increases;
wherein the body member includes a bottom liner forming the entire bottom surface of the body member and the attachment flaps each include a bottom flap liner forming the entire bottom surface of each of the attachment flaps;
wherein the bottom liner includes attachment surfacing that is configured to releasably attach to fabric and the bottom flap liners each include flap attachment surfacing that is configured to releasably attach to fabric;
wherein the attachment surfacing on the bottom liner extends longitudinally along the bottom liner;
wherein the bottom liner and the bottom flap liners are together formed from a single, continuous sheet of thin and flexible material;
wherein the body member includes a top liner and an absorbent layer positioned above the bottom liner and the attachment flaps include a top flap liner positioned above the bottom flap liner;
wherein the body member includes a tapering section adjacent to the rear end; and
wherein the top liner and the top flap liner are together formed from a single, continuous sheet of thin and flexible material.

21. The feminine hygiene pad of claim 20, wherein:
in the end section, the longitudinal sides of the body member are curved; and
in the main section, the longitudinal sides of the body member are curved.
